(19) United States
(12) Patent Application Publication
(10) Pub. No.: US 2017/0181884 A1
(43) Pub. Date: Jun. 29, 2017

(54) Hygienic Protective Barrier for Ostomy Bag

(71) Applicant: Lynn Vogel, Kimball, NE (US)

(72) Inventor: Lynn Vogel, Kimball, NE (US)

(21) Appl. No.: 15/389,733

(22) Filed: Dec. 23, 2016

Related U.S. Application Data
(60) Provisional application No. 62/271,433, filed on Dec. 28, 2015.

(51) Int. Cl. A61F 5/445

(52) U.S. Cl. CPC A61F 5/445

(57) ABSTRACT

A hygienic protective barrier. The hygienic protective barrier for use with an ostomy bag positioned between the bag and the user while in use, preventing direct contact between the bag and the user. The hygienic protective barrier includes a sheet made from moisture wicking material that provides protection from moisture or leakages and helps to absorb any sweat that might occur from usage of the ostomy bag. The sheet includes a cutout section that secures and closes around an opening of the ostomy bag via fasteners. The sheet can be removed and attached while an ostomy bag is affixed to a stoma. In one embodiment, the sheet is dimensioned to correspond to the size of a conventional ostomy bag.

### CROSS REFERENCE TO RELATED APPLICATION

This application claims the benefit of U.S. Provisional Application No. 62/271,433 filed on Dec. 28, 2015. The above identified paten, application is herein incorporated by reference in its entirety to provide continuity of closure.

### BACKGROUND OF THE INVENTION

### Field of the Invention

The present invention relates to a hygienic protective barrier that is affixed to an ostomy bag to prevent leaks from contacting the user. More specifically, the present invention provides a sheet that is connected to an ostomy bag and rests between the bag and the user while in use.

Devices have been disclosed in the known art that relate to ostomy bag assemblies. These include devices that have been patented and published in patent application publications. These devices in the known art have several drawbacks. For example, some of these devices attempt to prevent leaks and spills by providing improved valves and pads that protect a user's skin beneath the base plate of an ostomy port.

However, these known devices fail to provide a reusable protective barrier that affixes to an ostomy bag and provides a barrier for protecting against leaks along the entire length of the ostomy bag. Further, the known devices fail to provide a sheet that can easily be attached and removed from an ostomy bag operably connected to a stoma, without the need to disconnect the ostomy bag.

In light of the devices disclosed in the known art, it is submitted that the present invention substantially diverges in design elements from the devices in the known art and consequently it is clear that there is a need in the art for an improvement to existing hygienic ostomy devices. In this regard the instant invention substantially fulfills these needs.

### SUMMARY OF THE INVENTION

In view of the foregoing disadvantages inherent in the known types of hygienic ostomy device now present in the art, the present invention provides a new hygienic protective barrier for an ostomy hag wherein the same can be utilized for providing convenience for the user when utilizing an ostomy bag after surgery or other healthcare related incident.

The present invention provides a sheet connects to the ostomy bag between a base plate and the bag, and forms a barrier that prevents any leaks or spills from contacting the user. The sheet includes a first end having a first wing and a second wing forming a cutout section that receives and closes around an opening of the ostomy bag via fasteners. The sheet is thin enough to riot adversely affect normal function of the bag, while maintaining a sanitary environment. In one embodiment, the sheet is moisture wicking, thereby preventing sweat from affecting seal integrity between the ostomy bag and a stoma.

It is therefore an object of the present invention to provide a new and improved hygienic protective barrier for an ostomy bag that prevents leaks and spills from contacting a user's body or their clothes.

It is another object of the present invention to provide a hygienic protective barrier for an ostomy bag that can easily be attached and removed from an ostomy bag operably connected to a stoma, without the need to disconnect the ostomy bag from the stoma.

Another object of the present invention is to provide a hygienic protective barrier for an ostomy bag that is waterproof and washable, such that the present invention can be reused.

Yet another object of the present invention is to provide a hygienic protective barrier for an ostomy bag that is sized and dimensioned to correspond to a conventional ostomy bag.

Another object of the present invention is to provide a hygienic protective barrier for an ostomy bag that may be readily fabricated from materials that permit relative economy and are commensurate pith durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying, drawings wherein like numeral annotations are provided throughout.

FIG. 1 shows a front view of the hygienic protective barrier for an ostomy bag.

FIG. 2 shows a front view of the hygienic protective barrier for an ostomy bag in use.

FIG. 3 shows an exploded view of the hygienic protective barrier for an ostomy bag.

### DETAILED DESCRIPTION OF THE INVENTION

Reference is made herein to the attached drawings. Like reference numerals are used throughout the drawings to depict like or similar elements of the hygienic protective barrier for an ostomy bag. For the purposes of presenting a brief and clear description of the present invention, the preferred embodiment will be discussed as used for preventing ostomy bags from leaking or spilling onto a user or their clothes. The figures are intended for representative purposes only and should not be considered to be limiting in any respect.

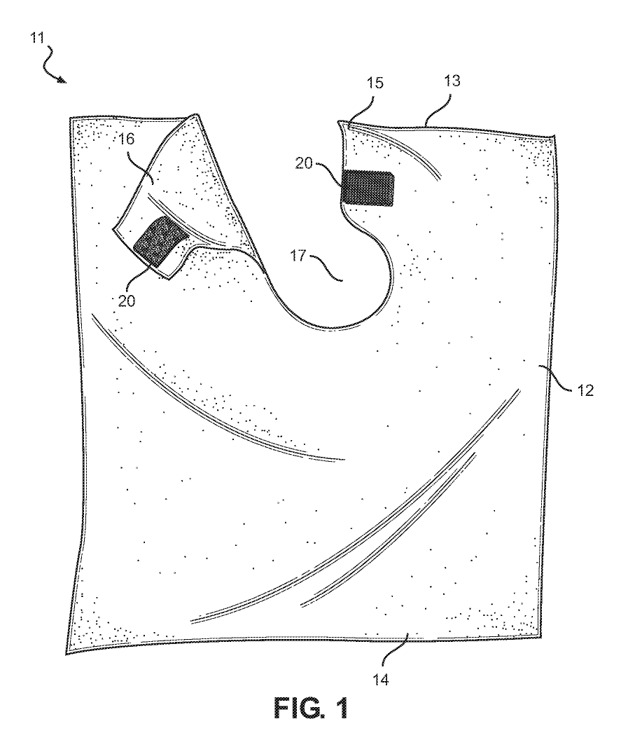

Referring to FIG. 1, there is shown a front view of the hygienic protective barrier for an ostomy bag. The hygienic protective barrier for an ostomy bag 11 comprises a sheet 12 having a First end 13 and a second end 14, wherein the second end 14 opposes the first end 13. The first end 13 includes a first wing 15 and a second wing 16 that form a cutout section 17 therebetween. In the shown embodiment, the first wing 15 and the second wing 16 have generally the same dimensions and are positioned equidistant from the lateral sides of the set 12. However, in alternative embodiments, the first and second wing 15, 16 may have asymmetrical shapes, wherein one or both wings 15, 16 extends in such a way that it substantially forms the perimeter of the cutout section 17.

In the shown embodiment, the first and second wing 15, 16 partially include overlapping sections, wherein fasteners 20 are disposed thereon. The fasteners 20 comprise mating fasteners disposed on the overlapping sections of each the first wing 15 and the second wing 16 for joining therewith. The mating fasteners are Velcro in the illustrated embodiment, however in alternative embodiments the fasteners 20 may comprise adhesives, buttons, and the like.

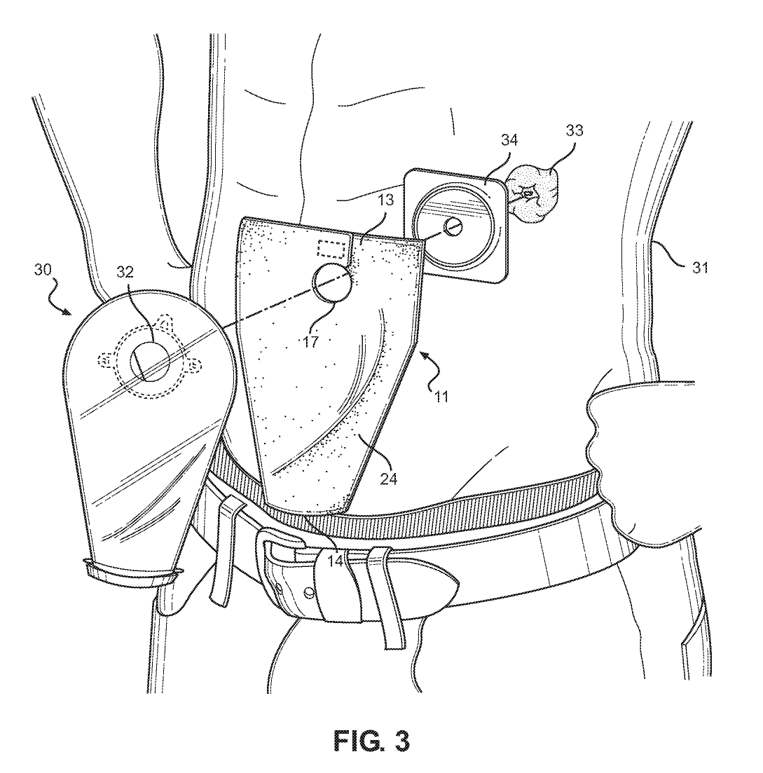

In the shown embodiment the first and second wings 15, 16 form the cutout section 17 that is movable between an open configuration and a closed configuration via the fastener 20. In the open configuration of the cutout section 17, the cutout section 17 is accessible from the first end 13. In the closed configuration of the cutout section 17, the cutout section 17 is not accessible from the first end 13. In this way, the cutout section 17 is positionable between a base plate of an ostomy bag and the bag of the ostomy bag (See FIG. 3). A perimeter of the cutout section 17 is configured to align with a perimeter of an opening of an ostomy bag.

The sheet 12 is preferable composed of waterproof material such as polyester. The sheet 12 is fie as it is adapted to generally conform to the shape of the ostomy bag and the user's body-when pressed thereagainst. In alternative embodiments, the material of the sheet 12 may include other fabrics that prevent the transmission of fluid thereacross. In one embodiment, the sheet 12 maintains a raw or unfinished edge so that the fabric can absorb any fluid efficiently. The unfished edge also alleviates discomfort from any bulky hem. In addition, the sheet 12 may comprise a moisture wicking material that prevents sweat from affecting seal integrity between the ostomy bag and a stoma.

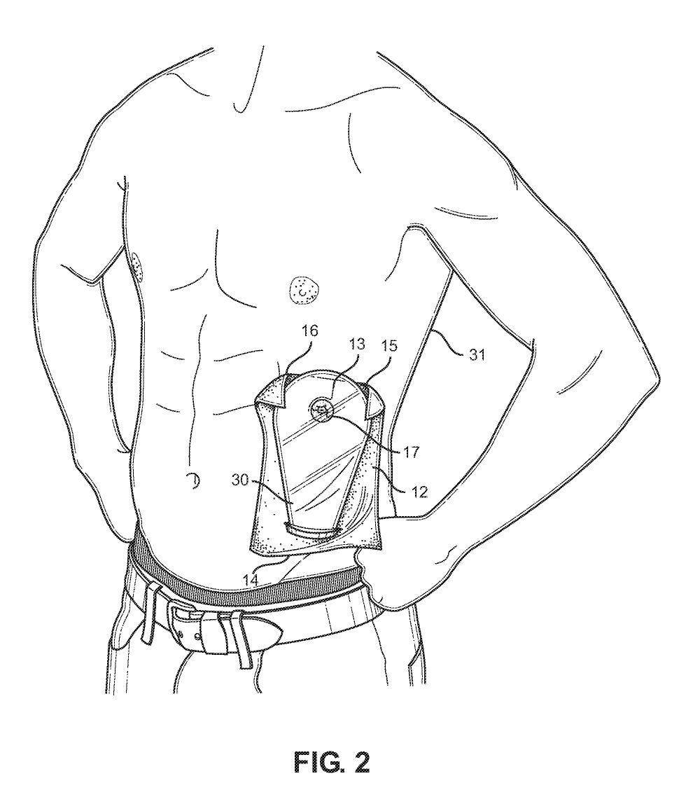

Referring to FIGS. 2 and 3, there is shown a front view of the hygienic protective barter for an ostomy bag in use and an exploded view of the hygienic protective barrier for an ostomy bag, respectively. In the shown views, the hygienic protective barrier 11 is positioned between a user's body 31 and the ostomy bag 30. If a leak is formed at the ostomy bag opening 32, or is the ostomy bag 30 itself becomes punctured, the sheet 12 provides a shield to prevent contact between the contents of the ostomy bag 30 and the user's body 31.

Specifically referring to FIG. 3, the sheet 12 is tapered from the first end 13 to the second end 14, wherein the distance across the sheet 12 decreases from the first end 13 to the second end 14. In one embodiment, the sheet 12 has a thickness between a first side 24 and an opposing second side of less than two millimeters. In this way, the sheet 12 is adapted to fit between the base plate 34 and the ostomy bag 30. The sheet 12 can be removed and attached to the ostomy bag 30, while the base plate 34 is affixed to the stoma 33.

In use, the hygienic protective barrier 11 is disposed between the base plate 34 and the ostomy bag 30. The base plate 34 provides a connection means with a stoma 33 of the body 31. The contents of the stoma 33 may be transferred to the ostomy bag 30 by going through the base plate 34 and the opening 32 of the ostomy bag 30, wherein the contents of the stoma 33 are deposited therein. The base plate 34 causes the stoma 33 to be in fluid communication with the opening 32. The cutout section 17 of the sheet 12 fits between the ostomy bag 30 and the body 31, such that the ostomy bag 30 does not directly contact any part of the body 31. In one embodiment, the distance between the first end 13 and the second end 14, and the distance between lateral sides of the sheet 12 are dimensioned to correspond to the ostomy bag 30 attached thereto.

It is therefore submitted that the instant on has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is riot desired to limit the invention to the exact construction and operation shown and described, and accordingly, all suitable modifications and equivalents may be resorted to, falling within the scope of the invention.

I claim:

1) A hygienic protective barrier for an ostomy bag, comprising:
a sheet comprising a first end and a second end, wherein the first end includes a first wing and a second wing;
a cutout section formed between the first wing and the second wing;
the cutout section movable between an open configuration and a closed configuration via a fastener;
wherein the open configuration of the cutout section is accessible from the first end; and
wherein the closed configuration of the cutout section is not accessible from the first end.

2) The hygienic protective barrier for an ostomy bag of claim 1, wherein a perimeter of the cutout section is dimensioned to align with a perimeter of an opening of an ostomy bag.

3) The hygienic protective barrier for an ostomy bag of claim 1, wherein the sheet is waterproof.

4) The hygienic protective barrier for an ostomy bag of claim 1, wherein the sheet comprises moisture wicking material.

5) The hygienic protective barrier for an ostomy bag of claim 1, wherein:
the sheet has a thickness between a first side and a second side of less than two millimeters.

6) The hygienic protective barrier for an ostomy bag of claim 1, wherein:
the fastener comprises mating fasteners disposed on the first wing and the second wing.

7) The hygienic protective barrier for an ostomy bag of claim 1, wherein:
the sheet is tapered from the first end to the second end.

8) The hygienic protective barrier for an ostomy bag of claim 1, wherein:
the sheet is dimensioned to correspond to an ostomy bag.

9) The hygienic protective barrier for an ostomy bag of claim 1, wherein:
the sheet can be either one of removed or attached between a base plate an ostomy bag while the ostomy affixed to a stoma.
